(19) United States
(12) Patent Application Publication
(10) Pub. No.: US 2025/0246270 A1
(43) Pub. Date: Jul. 31, 2025

(54) OPTIMIZED MOLECULE GENERATION WITH DISENTANGLED EQUIVARIANT REPRESENTATION

(71) Applicant: NEC Laboratories America, Inc., Princeton, NJ (US)

(72) Inventors: Renqiang Min, Princeton, NJ (US); Haoran Liu, College Station, TX (US)

(21) Appl. No.: 19/041,254

(22) Filed: Jan. 30, 2025

Related U.S. Application Data
(60) Provisional application No. 63/627,098, filed on Jan. 31, 2024.
(60) Provisional application No. 63/553,222, filed on Feb. 14, 2024.

(51) Int. Cl. G16C 20/50; G16C 20/30; G16C 20/70; G16C 20/80; G16H 20/10

(52) U.S. Cl. CPC G16C 20/50; G16C 20/30; G16C 20/70; G16C 20/80; G16H 20/10

(57) ABSTRACT

Methods and systems for three-dimensional (3D) molecule generation include training an autoencoder machine learning model that disentangles structural context of a molecule from properties of the molecule, using a loss function that further enforces equivariance of a coordinate representation and invariance of data likelihood. A 3D molecule is generated using the trained autoencoder machine learning model.

### RELATED APPLICATION INFORMATION

This application claims priority to U.S. Patent Application No. 63/627,098, filed on Jan. 31, 2024, and to U.S. Patent Application No. 63/553,222, filed on Feb. 14, 2024, each incorporated herein by reference in its entirety.

### BACKGROUND

### Technical Field

The present invention relates to molecule generation and, more particularly, to learning disentangled equivariant representations of molecules.

### Description of the Related Art

Drug discovery attempts to identify new three-dimensional (3D) molecular structures, within the vast space of possible chemicals, that have specific attributes. Traditional models have difficulty generating molecules with precise properties and controlling molecular attributes.

### SUMMARY

A method for three-dimensional (3D) molecule generation includes training an autoencoder machine learning model that disentangles structural context of a molecule from properties of the molecule, using a loss function that further enforces equivariance of a coordinate representation and invariance of data likelihood. A 3D molecule is generated using the trained autoencoder machine learning model.

A system for three-dimensional molecule generation includes a hardware processor and a memory that stores a computer program. When executed by the hardware processor, the computer program causes the hardware processor to train an autoencoder machine learning model that disentangles structural context of a molecule from properties of the molecule, using a loss function that further enforces equivariance of a coordinate representation and invariance of data likelihood, and to generate a 3D molecule using the trained autoencoder machine learning model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a machine learning model for generating three-dimensional (3D) molecules, in accordance with an embodiment of the present invention;

FIG. 2 is a block/flow diagram of a method for decoding latent features that specify structural context and properties of a 3D molecule, in accordance with an embodiment of the present invention;

FIG. 3 is a block/flow diagram of a method for training and using a model for generating 3D molecules, in accordance with an embodiment of the present invention;

FIG. 4 is a block diagram of a healthcare facility that can generate 3D molecules to tailor treatments to a patient, in accordance with an embodiment of the present invention;

FIG. 5 is a block diagram of a computing device that can generate 3D molecules, in accordance with an embodiment of the present invention;

FIG. 6 is a diagram of an exemplary neural network architecture that can be used to implement part of a molecule generation model, in accordance with an embodiment of the present invention; and

FIG. 7 is a diagram of an exemplary deep neural network architecture that can be used to implement part of a molecule generation model, in accordance with an embodiment of the present invention.

### DETAILED DESCRIPTION OF PREFERRED EMBODIMENTS

Molecule generation may be performed with a machine learning model that disentangles molecular properties from molecular structure, allowing each to be controlled independently. The disentangled representation effectively separates a generative model's latent space into property and structure aspects. An autoencoder model may be used with a coordinate prediction loss function to generate new molecules. The autoencoder model provides property-guided generation and structure-guided generation.

To this end, the latent space is factorized into the property and structure context of 3D molecules. This disentanglement is performed using a Wasserstein regularization loss that forces the independence between property latent variables and structure latent variables. A prediction head ensures that property latent variables have property-relevant information. This ensures explicit control over molecule attributes.

The model may have two distinct generation modes, including property-guided generation for generating molecules with specific property values and structure-guided generation for modifying molecular attributes while preserving overall molecular structure. The coordinate loss function with structure alignment guarantees equivariance of the coordinate representation and invariance of data likelihood.

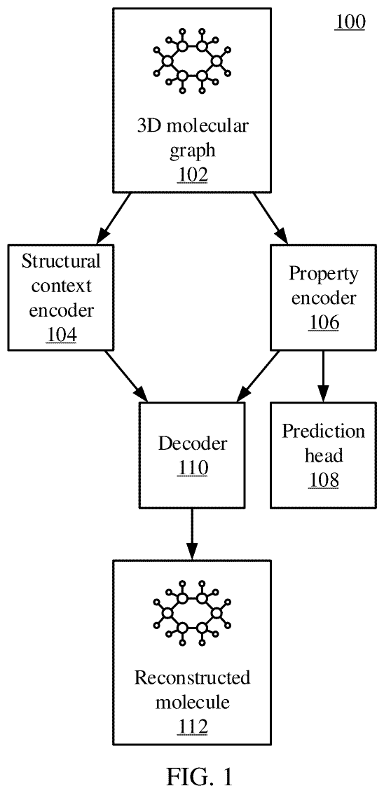

Referring now to FIG. 1, an autoencoder framework is shown. A 3D molecular graph 102 is input to the autoencoder. The graph 102 may include, for example, nodes that represent atoms that make up the molecule, with edges that represent bonds between the atoms. The autoencoder 100 includes two distinct encoders, a structural context encoder 104 and a property encoder 106. The two encoders create feature vectors of the 3D molecular graph 102 in their respective latent spaces, with the structural context encoder 104 encoding structural features and with the property encoder 106 encoding property features. The encoders may be implemented as E(3)-equivariant graph neural networks (GNNs).

The encoded features are processed jointly by a decoder 110, which may also be implemented as an E(3)-equivariant GNN. The decoder 110 outputs a reconstructed molecule (during training) or a new molecule having desired structure and properties (during runtime). The property features are separately processed by a prediction head 108, which may be implemented as a multi-layer perceptron (MLP), to verify the property or properties that they encode.

An auto-regressive, fragment-based 3D generation may be used to generate large-scale, drug-like molecules that may be suitable for use as pharmaceuticals. The space of 3D molecular graphs may be expressed as [symbol] , where each 3D molecula graph G∈ [symbol] includes the fragment node set [symbol] , the edge set ε, and the fragment coordinate matrix [symbol] . Each fragment represents a combination of several atoms and bonds. For example, a benzene ring may be represented as a fragment that includes six carbon atoms and aromatic bonds. Each fragment i∈ [symbol] is associated with a node feature vi and a position vector rᵢ which corresponds to the i^{th} line of [symbol] and represents the center coordinates of the fragment. The edge eᵢⱼ∈ε indicates that two fragments i,j∈ [symbol] share a bond/atom.

This fragmentization includes assembled rules for neighboring fragments and enables a model of a substantial portion of the chemical space with a reasonably sized fragment vocabulary. The generation process may be iterative, a next graph state at each step t is predicted as [symbol] ₜ₊₁= [symbol] ( [symbol] ), where [symbol] is the auto-regressive generation model. The model provides explicit control over molecular attributes while ensuring both E(3)-invariance and equivariance properties.

A generative model with explicit control is defined as [symbol] : [symbol] ₁× [symbol] ₂→ [symbol] , where [symbol] ₁ and [symbol] ₂ represent the attribute spaces of two different attributes and where [symbol] is the space of generated objects. The model [symbol] is considered to have explicit control if, for any given attributes [symbol] ₁∈ [symbol] ₁ and [symbol] ₂∈ [symbol] ₂, it consistently generates an object x∈ [symbol] reflecting those attributes. In the present model, [symbol] ₁ is the space of target molecular properties and [symbol] ₂ is the structural context of molecules. Manipulating [symbol] ₁ allows for altering molecular properties molecular properties and maintaining their structural context, whereas adjusting [symbol] ₂ enables refinement of molecule structures while maintaining their chemical properties.

The 3D Euclidean group (abbreviated herein as E(3)) is the group generated by all 3D rotations, translations and reflections in 3D space. In the context of 3D molecule generation without an external reference structure, the model maintains equivariance of coordinate representation and invariance of data likelihood. First, the coordinates of a molecule's nodes are equivariant to the positions of other nodes in the 3D molecule. That means, when generating the coordinates rₜ for an atom at iteration t, if the current input structure [symbol] ₜ is rotated or translated, then rᵢ needs to be rotated or translated correspondingly. Formally, consider a rotation matrix R∈SO(3) and a translation vector τ∈ [symbol] ³, for the coordinate generation model [symbol] ^{r}, Rrₜ+τ= [symbol] ^{r}(R [symbol] ₜ+τ). In contrast, the likelihood π(rᵢ| [symbol] ₜ) should be invariant to rotations and translations as they do not change the 3D structure, so that π(Rrₜ+τ|R [symbol] ₜ+τ)=π(rₜ| [symbol] ₜ), for any rotation R∈ [symbol] ^{3×3} or translation τ∈ [symbol] ³.

The present model may therefore be implemented as an E(3)-equivariant Wasserstein autoencoder (E3WAE) model, where the generation of the 3D molecule is factorized into two disentangled factors: the property and the structural context. The former variable comprises the chemical property of the 3D molecule, while the latter refers to all other 3D structure patterns that do not relate to the property yet reflect the chemical constraints within the molecular chemical space. This factorization allows for explicit control during the generation process.

The encoders 104 and 106 extract invariant and equivariant latent variables, respectively:

z_{h,e},z_{y,e}=Θₑ( [symbol] ),

where e={p, s} represents “property” or “structural context.” The invariant latent variable associate with node i, z_{h,e}ⁱ∈z_{h,e}, has a dimensionality of dₕ. Correspondingly, the equivariant latent variable z_{v,e}ⁱ∈z_{v,e} is dimensioned in [symbol] ^{d}^{_{v×3}}.

In some embodiments, an E(3)-equivariant vector neurons multilayer perceptron (VN-MLP) and mixed-features message passing (MF-MP) may be used as building blocks for both branches of encoders Θe. These are effective in integrating both invariant and equivariant features, ensuring the property space's invariance and the coordinate space's equivariance. However, it is important to note that our model is compatible with any E(3)-equivariant architecture.

For the property branch, the property latent variables z_{h,p} and z_{v,p} are first derived by the property encoder Θₚ(G). Subsequently, a Readout function is used to obtain graph-level representations. The Readout function is implemented as either the average or the summation of all node embeddings. To ensure that these latent variables carry information related to the property of 3D molecules, the auxiliary prediction head 108 is expressed as [symbol] ₚᵣₒₚ and takes the aggregated z_{h,p} as input and predicts the target property value of the 3D molecule:

ŷ= [symbol] ₚᵣₒₚReadout(z_{h,p})),

For the structure context branch, the context latent variables z_{h,s} and z_{v,s} are encoded using the structural encoder Θₛ(G). To ensure that these variables capture comprehensive information about the molecule, specifically excluding patterns related to the target property, an autoregressive molecule reconstruction loss.

A Wasserstein autoencoder regularization loss may be used to achieve the disentanglement between property and context latent variables. This approach involves minimizing the Maximum Mean Discrepancy (MMD) between the distribution of latent variables and an isotropic multivariate Gaussian prior, denoted as z˜P_{z}. Specifically, for the invariant latent variables zₕ˜Q_{z}^{h}, where zₕ=concat(z_{h,p}, z_{h,s}), and an isotropic Gaussian prior p_{z}^{h}= [symbol] (0, I_{2d}), the disentanglement loss for invariant variables is computed as

[symbol]

_{Dis}=MMD(p_{z}^{h},Q_{z}^{h}).

For the equivariant latent variables, zᵥ=concat(z_{v,p}, z_{v,s}) and zᵥ∈ [symbol] ^{2d}^{_{v×3}}, independence is maintained along the 2dᵥ axis while allowing for covariance along the remaining dimension. To this end, three isotropic Gaussian priors are sampled from p^{v}=N(0, I₂) to calculate the corresponding disentanglement Wasserstein loss as:

[symbol]

_{Dis}=MMD(P_{z}^{v},Q_{z}^{v}),

where p^{v}∈ [symbol] ^{2d}^{_{v×3}} is the combined Gaussian priors and Q_{z}^{v}∈ [symbol] ²^{₀}^{d}^{_{v×3}} is the distribution of the equivariant latent variables zᵥ. The total disentanglement loss is then calculated as the sum of these components: [symbol] _{Dis}= [symbol] _{Dis}^{h}+ [symbol] _{Dis}^{v}.

For MMD estimation, an input batch of latent variables {zᵢ}_{i=1, . . . ,m} may have batch size m. Corresponding samples {\tilde{z}ᵢ}_{i=1, . . . ,m} are randomly drawn from the Gaussian prior, {\tilde{z}ᵢⱼᵢ}, . . . , . . . , m, maintaining the same sample size. The MMD is then calculated using a linear time unbiased estimator as

$$MMD ( {P}_{z} , {Q}_{z} ) = \text{} \frac{1}{⌊ m / 2 ⌋} {\sum }_{i = 1}^{⌊ m / 2 ⌋} [ k ( {z}_{2 i - 1} , {z}_{2 i} ) + k ( {\tilde{z}}_{2 i - 1} , {\tilde{z}}_{2 i} ) - k ( {z}_{2 i - 1} , {\tilde{z}}_{2 i} ) - k ( {z}_{2 i} , {\tilde{z}}_{2 i - 1} ) ]$$

where k is the kernel function, implemented using a radial basis function (RBF) kernel with σ=1.

Minimizing this loss aligns the joint distribution of the latent embeddings with the isotropic normal distributions, so that the property and context latent variables are independent. Additionally, the Gaussian shape of the latent space facilitates smooth interpolation, effective regularization, and enhanced generation diversity.

The decoder [symbol] ( [symbol] |zₕ, zᵥ) reconstructs 3D molecule graph [symbol] fragment-by-fragment in an auto-regressive manner. Specifically, the decoder 110 may be implemented as another E(3)-equivariant GNN. The connectivity rules between fragments are incorporated by masking out invalid edges. The present model uses fragments as nodes and makes use of a coordinate prediction loss that optimizes equivariant networks. This approach enables the generation of molecules using equivariant networks without relying on any external conditioning, such as reference structures like linker designs and pocket-based generation.

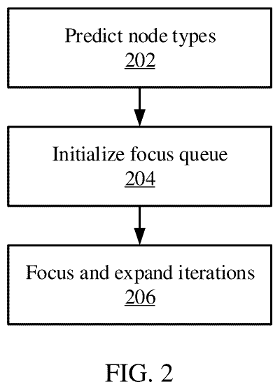

Referring now to FIG. 2, a method of a decoding process is shown. The decoding process includes node type prediction 202, focus queue initialization 204, and focusing and expanding iterations 206. Initially, fragment types {xᵢ}ᵢ₌₁ⁿ for all n nodes in [symbol] are determined from latent variables zₕ and zᵥ. The fragment type logits are obtained from latent variables through a self-attention mechanism and an MLP, and these logits are then used to sample all fragment types. Once the node types are sampled, their embeddings are concatenated by block 202 with the corresponding latent variables zₕⁱ for subsequent steps.

Block 204 initializes a focus queue Q with a randomly chosen node from [symbol] . For each focus node f popped from Q in block 206, the decoder 110 predicts an expand edge connecting f to another node. If the connected node u is not a stop node or being linked for the first time, its coordinate (xᵤ,yᵤ,zᵤ) is predicted. Node u is then added to Q if unvisited. This process repeats until a stop node is reached, marking f as visited. The order of node focus and edge connection may be determined using a breadth-first search, facilitating teacher-forcing training. The reconstruction process ends when Q is empty, ensuring that all nodes in the connected component of [symbol] have been considered for expansion.

Concretely, in each iteration t of the focus and expand phase 206, the currently reconstructed subgraph is [symbol] ₜ=( [symbol] ₜ, εₜ, [symbol] ₜ). Initially, the latent node embeddings are updated as \hat{z}ₕ, \hat{z}ᵥ with an MF-MP layer. Then the edge logits between the focus node f and any node i are obtained with

$${e}_{f , i} = Φ ( {\hat{z}}_{h}^{f} , {\hat{z}}_{h}^{i} , ‖ {\hat{z}}_{v}^{f} ‖ , ‖ {\hat{z}}_{v}^{i} ‖ , {m}_{f , i} , {\sum }_{j ∈ {v}_{t}} {\hat{z}}_{h}^{j} , {\sum }_{j ∈ {v}_{t}} {\hat{z}}_{h}^{j} )$$

where Φ is a feed forward network and m_{f,i}∈{0,1} indicates whether fragments f and i can be connected, based applied to the edge logits to determine the probabilities for each edge. Node u is then determined by identifying which node i has the highest probability.

To predict the coordinate for the newly connected node u, the geometric center r̅ₜ of current subgraph [symbol] ₜ is taken as a reference point to predict a displacement of node u related to the reference point. Initially, two sets of pair-wise interactions are predicted in the current connected graph as of pair-wise interactions in the current connected graph as

pᵢⱼ^{(k)}=Ψ(\hat{z}ₕⁱ,zₕⁱ, [symbol] W^{(k)}\hat{z}ᵥⁱ,W^{(k)}\hat{z}ᵥ^{j} [symbol] ),k=1,2

where Ψ is a feed forward network and W^{(k)} is a learnable linear transformation. The displacement of node u is then calculated by:

$${d}_{u} = {\sum }_{j ∈ {v}_{t}} {p}_{u j}^{( 1 )} ( {r}_{j} - {\overline{r}}_{t} ) + {Ω}_{1} ( {\sum }_{j ∈ {v}_{t}} {p}_{u j}^{( 2 )} {Ω}_{2} ( {\hat{z}}_{v}^{u} , {\hat{z}}_{v}^{j} ) )$$

where Ω₁ and Ω₂ are VN-MLP layers. Finally, the predicted coordinates of node u is obtained by \hat{r}ᵤ=dᵤ+r̅ₜ.

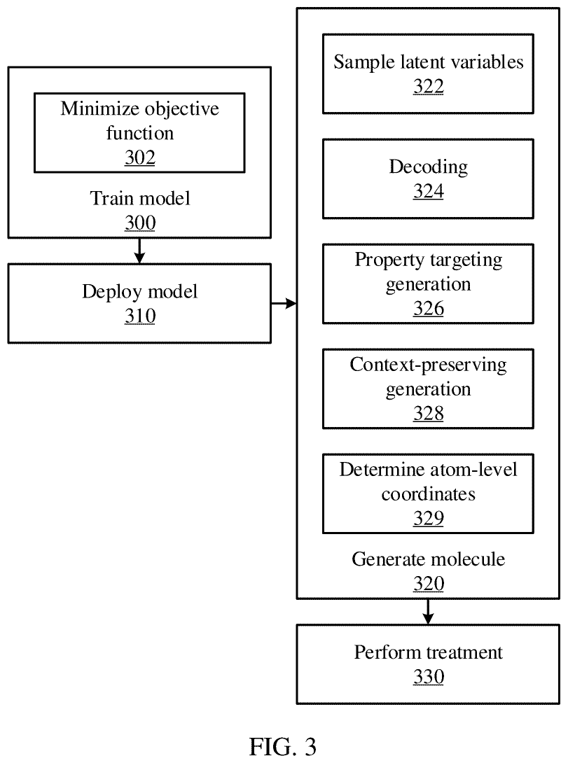

Referring now to FIG. 3, a method for training and using a 3D molecule generating model is shown. Block 300 trains the model, for example by modifying parameters of the autoencoder to minimize an objective function using a training dataset. The training dataset may be obtained from public benchmark datasets or private data. Once the model has been trained, it may be deployed 310 to a target environment. In some cases the model may be implemented in the same system that is used to train it, while in others it may be deployed to one or more target environments, where it will be used to generate new molecules in accordance with input structure and/or properties.

Once deployed, block 320 generates a new molecule. In some cases the new molecule may be generated to achieve specified properties while remaining close to some input structure. In some cases a wholly new structure may be generated. Block 330 then performs an action using the new molecule. For example, block 330 may include synthesizing the molecule to create a drug, which may then be administered to a patient as a treatment for some condition. Once the molecular composition and 3D structure of a molecule is known, it can be readily manufactured.

During training 300, the model may be trained by minimizing a weighted sum of three individual losses:

$${ℒ}_{Total} = {ℒ}_{Prop} + {αℒ}_{D i s} + {βℒ}_{R e c o n}$$

where α and β are the trade-off weights for the losses. Specifically, [symbol] _{Dis} is the disentanglement loss described above. An L1 loss may be used as the property prediction loss for the auxiliary property prediction head [symbol] ₚᵣₒₚ attached to property encoder Θₚ

[symbol]

_{Prop}=∥y−ŷ∥₁

where y denotes the ground truth value of the target property. Finally, for the decoder 110, a reconstruction loss is used which may itself include three parts:

$${ℒ}_{Recon} = {ℒ}_{NodeType} + {ℒ}_{Edge} + {ℒ}_{Coords}$$

Initially, a cross-entropy loss [symbol] _{NodeType} is used for a classification task to accurately determine the types of nodes. Following this, another cross-entropy loss, [symbol] _{Edge}, is applied to predict the edges in each iteration of the process. Finally, a log-Mean Squared Error (MSE) loss is used as the coordinate prediction loss [symbol] _{Coords} at each iteration t:

$${ℒ}_{C o o r d s}^{t} = log ( {\sum }_{t = 1}^{m} {f}_{i} {‖ {\overline{r}}_{i} - {r}_{i} ‖}^{2} ) / ( {\sum }_{t = 1}^{m} {f}_{i} )$$

where fᵢ is a binary flag indicating the presence of a newly added node in the i-th subgraph [symbol] ₜ⁽ⁱ⁾ that is not a stop node.

However, log-MSE loss cannot be applied directly. For the first iteration t=1, there are only two nodes in the current subgraph, in which ∥ [symbol] ₜ∥=2 and [symbol] ₜ∈ [symbol] ^{2×3}. The only constraint to the current 3D structure is the distance between the two existing nodes. Thus, the ground truth coordinates can be considered as any point on a sphere with distance dₜ=∥rᵤ−r̅ₜ∥ to the reference point coordinate r̅ₜ=r_{f}, where rᵤ denotes the ground truth coordinate of newly added node u and r_{f} is the coordinate of the focus and the only node f in previous subgraph.

Moreover, for the situation that there are three nodes in the current subgraph, where ∥ [symbol] ₜ∥=3 and [symbol] ₜ∈ [symbol] ^{3×3}, the ground truth coordinate is considered equivalent to any point with a distance dₜ=∥rᵤ−r̅ₜ∥ to r̅ₜ and a angle θₜ=arccos (sₜ, s_{uf}). Here sₜ, s_{uf} are used to denote the unit vectors of rᵤ−r̅ₜ and rᵤ−r_{f}, respectively. Therefore, using the log-MSE coordinate loss directly neglects all these situations and violates the symmetric-invariance property of the geometric space.

To adapt E(3)-equivariant networks to de-novo molecule generation task without external reference structure, the coordinates may be aligned with a Kabsch algorithm and then the coordinate loss may be calculated with transformed coordinates for the case when there are fewer than or equal to three nodes in any samples in this batch. A rotation matrix R∈SO(3) and a translation vector τ∈ [symbol] ³ may be calculated for the maximum alignment between generated coordinates [symbol] ₜ=[\hat{r}ᵢ]_{i∈v}_{ₜ} and ground truth coordinates [symbol] ₜ. Then transformed coordinates of generated nodes are obtained by

$${\tilde{r}}_{i} = R {\hat{r}}_{i} + τ , ∀ i ∈ {V}_{t}$$

Thus, for samples with three or fewer nodes at the current iteration, the coordinate loss is:

$${ℒ}_{C o o r d s}^{t , i} = {\sum }_{j ∈ {V}_{t , i}} {‖ {\tilde{r}}_{j} - {r}_{j} ‖}^{2}$$

During molecule generation 320, latent variables zₕ and zᵥ are sampled 322 and the maximum number of fragments is set to N. Note that the exact number of fragments might be smaller than N. Then the generation process performs decoding 324, for example as described above in FIG. 2, but differs by operating without teacher forcing, relying solely on the model's self-guided predictions. Notably, the disentangled latent space which enables explicit control over two key aspects of molecule generation.

For property-targeting generation 326, predefined property latent variables z_{h,p}>z_{v,p} are combined with sampled or template context latent variables z_{h,s}, z_{v,s}. This combination is then fed into the decoder 110, producing new molecules [symbol] = [symbol] ( [symbol] |z_{h,p}, z_{v,p}) with targeted properties. For context-preserving generation 328, predefined context latent variables z_{h,s}, z_{v,s} are combined with either sampled or template property latent variables z_{h,s}, z_{v,s}. Using such latent variables, the decoder 110 can generate new molecules [symbol] = [symbol] ( [symbol] |z_{h,s}, z_{v,s}) that refine certain properties while maintaining the core molecule framework. The predefined latent variables are obtained either by directly using or by performing interpolation or extrapolation with latent variables from existing molecules.

Once the 3D molecular graph is complete at the fragment level, including all fragment types, links, and center coordinates, block 329 determines the atom-level coordinates. Block 329 picks a fragment and explores all feasible links to its neighbors, selecting the one nearest to a pre-defined fragment center. Each potential connection may be modeled, evaluating their suitability based on root-mean-square deviation (RMSD) from the center. This procedure is iteratively applied to build the structure, fragment by fragment. Finally, these local structures may be aligned within the molecular framework using the Kabsch algorithm, adjusting the coordinates to match target positions.

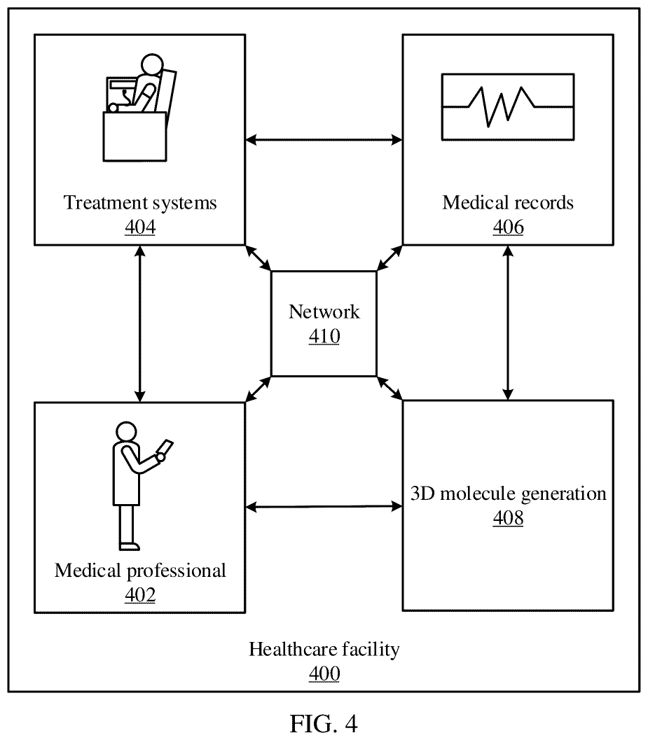

Referring now to FIG. 4, a diagram of information extraction is shown in the context of a healthcare facility 400. 3D molecule generation 408 may be used to generate a custom treatment for a patient, in accordance with target properties and structure. The 3D molecule generation 408 may be used to generate such a molecule responsive to a patient's medical condition based on up-to-date medical records 406.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Medical professionals 402 may use 3D molecule generation 408 to provide customized healthcare that is tailored to the patient's needs. For example, the medical professionals 402 may use 3D molecule generation 408 to generate a new drug that is similar to known-effective drugs, but that avoids a patient's allergies or other sensitivities.

The different elements of the healthcare facility 400 may communicate with one another via a network 410, for example using any appropriate wired or wireless communications protocol and medium. Thus the 3D molecule generation 408 can be used to design a treatment that targets a patient's specific condition, for example using test results and medical records 406. The treatment systems 404 may be used to generate and administer a therapy based on 3D molecule generation 408.

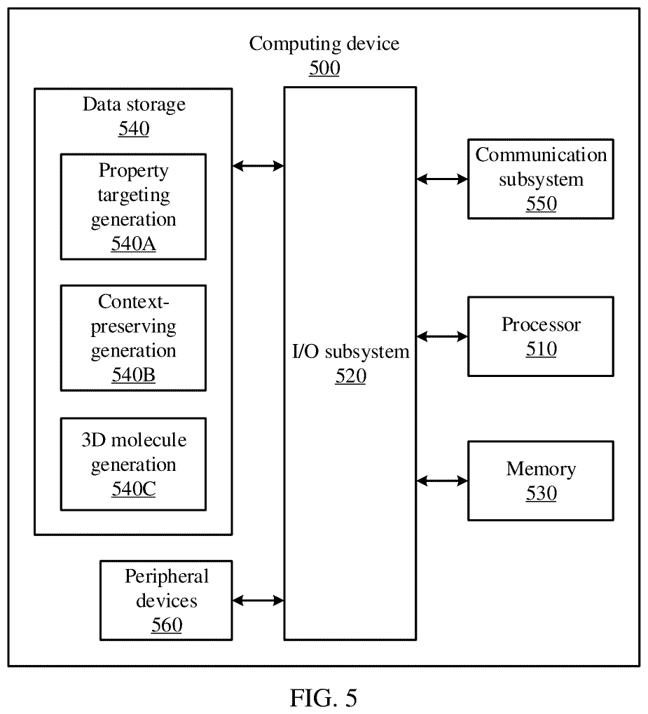

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for property targeting generation, 540B for context-preserving generation, and/or 540C for 3D molecule generation. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

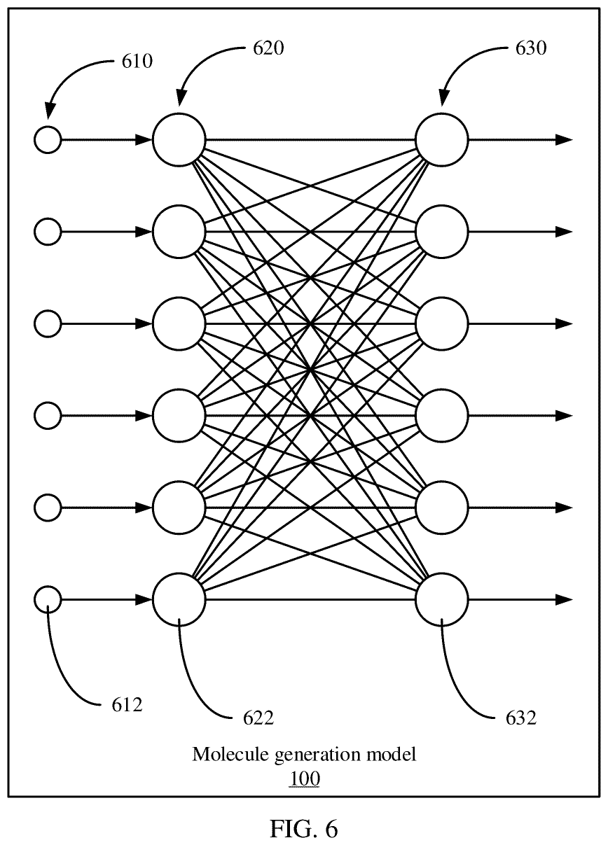

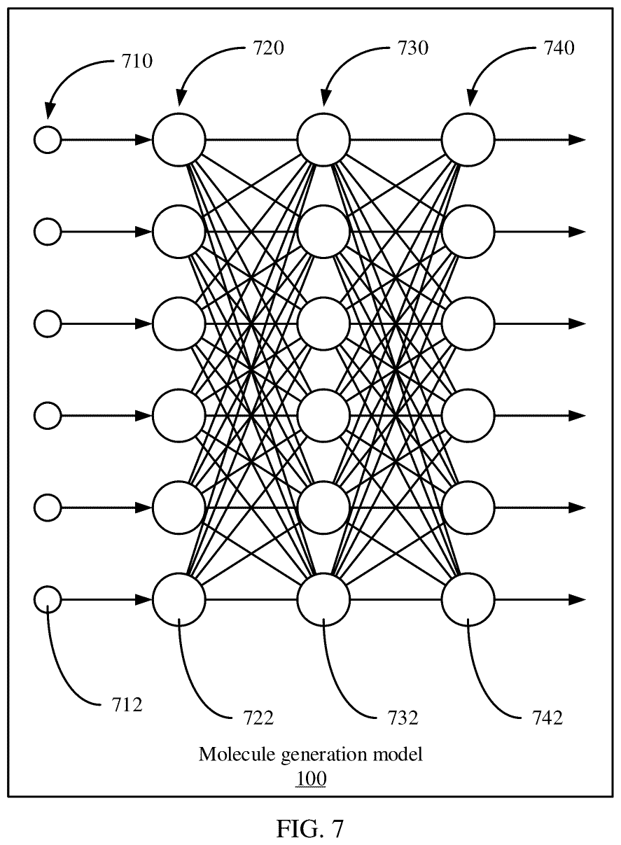

Referring now to FIGS. 6 and 7, exemplary neural network architectures are shown, which may be used to implement parts of the present models, such as the molecule generation model 100. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . ,wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for three-dimensional (3D) molecule generation, comprising:
training an autoencoder machine learning model that disentangles structural context of a molecule from properties of the molecule, using a loss function that further enforces equivariance of a coordinate representation and invariance of data likelihood; and
generating a 3D molecule using the trained autoencoder machine learning model.

2. The method of claim 1, wherein the autoencoder machine learning model includes a structural context encoder and a property encoder that each process a molecular graph input to generate respective features.

3. The method of claim 2, wherein the autoencoder machine learning model further includes a prediction head that verifies a property encoded by the property encoder.

4. The method of claim 2, wherein the autoencoder machine learning model further includes a decoder that outputs a new 3D molecule graph.

5. The method of claim 1, wherein generating the 3D molecule includes property targeting generation that specifies one or more properties of the 3D molecule.

6. The method of claim 1, wherein generating the 3D molecule includes context-preserving generation that preserves structural context of an input molecule.

7. The method of claim 1, wherein the autoencoder machine learning model is implemented using a plurality of E(3)-equivariant graph neural networks.

8. The method of claim 1, wherein the loss function is:
$${ℒ}_{Total} = {ℒ}_{Prop} + {αℒ}_{D i s} + {βℒ}_{R e c o n}$$
where [symbol] ₚᵣₒₚ is a property prediction loss, [symbol] _{Dis} is a disentanglement loss, [symbol] _{Recon} is a reconstruction loss, and α and β are weighting parameters.

9. The method of claim 8, wherein the reconstruction loss includes:
$${ℒ}_{Recon} = {ℒ}_{NodeType} + {ℒ}_{Edge} + {ℒ}_{Coords}$$
where [symbol] _{NodeType} is a cross-entropy loss to determine node types, [symbol] _{Edge} is a cross-entropy loss to predict edges, and [symbol] _{Coords} is a coordinate prediction loss expressed as:
$${ℒ}_{C o o r d s}^{t , i} = {\sum }_{j ∈ {V}_{t , i}} {‖ {\tilde{r}}_{j} - {r}_{j} ‖}^{2}$$
where rⱼ is a coordinate of a node j, \tilde{r}ⱼ is a transformed coordinate of a node j, and [symbol] _{t,i} is a set of fragments at an iteration t.

10. The method of claim 1, further comprising administering a drug based on the 3D molecule to a patient that includes properties tailored to the patient.

11. A system for three-dimensional (3D) molecule generation, comprising:
a hardware processor; and
a memory that stores a computer program which, when executed by the hardware processor, causes the hardware processor to:
train an autoencoder machine learning model that disentangles structural context of a molecule from properties of the molecule, using a loss function that further enforces equivariance of a coordinate representation and invariance of data likelihood; and
generate a 3D molecule using the trained autoencoder machine learning model.

12. The system of claim 11, wherein the autoencoder machine learning model includes a structural context encoder and a property encoder that each process a molecular graph input to generate respective features.

13. The system of claim 12, wherein the autoencoder machine learning model further includes a prediction head that verifies a property encoded by the property encoder.

14. The system of claim 12, wherein the autoencoder machine learning model further includes a decoder that outputs a new 3D molecule graph.

15. The system of claim 11, wherein the computer program further causes the hardware processor to perform property targeting generation that specifies one or more properties of the 3D molecule.

16. The system of claim 11, wherein the computer program further causes the hardware processor to perform context-preserving generation that preserves structural context of an input molecule.

17. The system of claim 11, wherein the autoencoder machine learning model is implemented using a plurality of E(3)-equivariant graph neural networks.

18. The system of claim 11, wherein the loss function is:
$${ℒ}_{Total} = {ℒ}_{Prop} + {αℒ}_{D i s} + {βℒ}_{R e c o n}$$
where [symbol] ₚᵣₒₚ is a property prediction loss, [symbol] _{Dis} is a disentanglement loss, [symbol] _{Recon} is a reconstruction loss, and α and β are weighting parameters.

19. The system of claim 18, wherein the reconstruction loss includes:
$${ℒ}_{Recon} = {ℒ}_{NodeType} + {ℒ}_{Edge} + {ℒ}_{Coords}$$
where [symbol] _{NodeType} is a cross-entropy loss to determine node types, [symbol] _{Edge} is a cross-entropy loss to predict edges, and [symbol] _{Coords} is a coordinate prediction loss expressed as:
$${ℒ}_{C o o r d s}^{t , i} = {\sum }_{j ∈ {V}_{t , i}} {‖ {\tilde{r}}_{j} - {r}_{j} ‖}^{2}$$
where rⱼ is a coordinate of a node j, \tilde{r}ⱼ is a transformed coordinate of a node j, and [symbol] _{t,i} is a set of fragments at an iteration 1.

20. The system of claim 11, wherein the computer program further causes the hardware processor to trigger administration of a drug based on the 3D molecule to a patient that includes properties tailored to the patient.
